(19) United States
(12) Patent Application Publication
(10) Pub. No.: US 2009/0043168 A1
(43) Pub. Date: Feb. 12, 2009

(54) Self-releasing tourniquet and method

(75) Inventor: Anto Parambil, Sugar Land, TX (US)

Correspondence Address: SCOTT DAVID COMPTON, #593, 2386 South Dairy Ashford Road, Houston, TX, 77077, US

(73) Assignee: Israel Ramos, Katy, TX (US)

(21) Appl. No.: 11/891,186

(22) Filed: Aug. 9, 2007

(51) Int. Cl. A61B 17/00

(52) U.S. Cl. 600/203

(57) ABSTRACT

A self-releasing tourniquet comprising a compression means and a releasing means, whereby the releasing means is configured to fail without any external influence at a predetermined time following application of the tourniquet.

### CROSS-REFERENCE TO RELATED APPLICATIONS

Not applicable.

### STATEMENT REGARDING FEDERALLY SPONSORED RESEARCH OR DEVELOPMENT

Not applicable.

### FIELD OF THE APPLICATION

This application relates generally to tourniquets used by health care professionals.

### BACKGROUND

A tourniquet is a constricting or compressing device commonly used by health care professionals to control venous and arterial circulation to an extremity of a patient for a period of time. Typically, tourniquets are applied to extremities of patients to inhibit the flow of blood in order to start an IV line, to stop bleeding, or to draw blood from a patient, among other reasons.

A typical tourniquet used by health care professionals today includes a strip of material that can be tightly wrapped and tied around a patient's extremity to apply pressure circumferentially upon the skin and underlying tissues of the extremity to stop the flow of blood there through. Although effective, if a health care professional leaves the tourniquet on the extremity in a compressed state too long, or forgets to remove the tourniquet altogether, the prolonged restriction of blood flow may result in ischemia or other injury to the patient, including for example, circulatory damage, neurological damage, vascular damage, and/or muscular damage.

A self-releasing tourniquet is desired that can automatically release itself from a patient after a predetermined amount of time.

### SUMMARY

The present application provides a self-releasing tourniquet comprising: a compression means; and a releasing means positioned at a non-terminal point along the length of the compression means, thereby dividing the compression means into two parts; whereby the releasing means is configured to fail at a predetermined time following application of the tourniquet.

The present application also provides a self-releasing tourniquet comprising: a first compression section; a second compression section; a foldable releasing means attached to the first compression section at a first end and attached to the second compression section at a second end; an adhesion means for adhering the first compression section to the second compression section prior to application of the tourniquet; and an activation means located on the first compression section and configured to contact the releasing means upon application of the tourniquet, the activation means being effective to trigger the failure of the releasing means.

The present application also provides a method of disabling a tourniquet during use, the method comprising the following steps: providing a tourniquet comprising a compression means, a releasing means, an adhesion means, and an activation means; applying the tourniquet to an extremity of a patient thereby contacting the activation means to the releasing means; forming a reaction between the activation means and the releasing means which results in the failure of the releasing means; and decompressing the tourniquet from the extremity of the patient.

### BRIEF DESCRIPTION OF THE FIGURES

FIG. 1 illustrates a simplified embodiment of an unused tourniquet including a compression means and a releasing means.

FIG. 2 illustrates a perspective view of an unused tourniquet including an adhesion member and activator located on the first compression section.

FIG. 3A illustrates a side view of a simplified embodiment of an unused tourniquet.

FIG. 3B illustrates a side view of a simplified embodiment of a tourniquet during activation as the overlapping section is released from the second compression section.

FIG. 3C illustrates a side view of a simplified embodiment of a tourniquet during operation including an activator in contact position with the safety member.

FIG. 3D illustrates a side view of a simplified embodiment of a tourniquet in a self-released state following compression of the tourniquet to a patient.

FIG. 4A illustrates a top view of a simplified embodiment of an unused tourniquet prior to application including a phantom view showing the initial location of an adhesion member and an activator of the first compression section contacting the second compression section.

FIG. 4B illustrates a top view of a simplified embodiment of a tourniquet in a compressed position including a phantom view showing the location of the activator contacting the releasing means.

### DESCRIPTION

It has been found that a tourniquet can be applied to an extremity of an individual to restrict blood flow through the extremity for a period of time and then (1) release itself, (2) automatically decompress, or (3) otherwise detach itself from the extremity without any external influence. Heretofore, such a desirable achievement has not been considered possible, and accordingly, the novel tourniquet and method of this application measure up to the dignity of patentability and therefore represent a patentable concept.

Before describing the invention in detail, it is to be understood that the present tourniquet and method are not limited to particular embodiments. It is also to be understood that the terminology used herein is for the purpose of describing particular embodiments only, and is not intended to be limiting. The phrase “self-releasing” or “self-releasable” refers to the automatic removal, decompression, loosening or detachment of the tourniquet from the extremity of an individual. The phrase “self-activated”, “self-activating”, and variances thereof, refers to the triggering of the self-releasing means of the tourniquet during operation of the tourniquet. The phrase “external influence” herein refers to external tampering, controlling or manual manipulation of the tourniquet. The terms “apply”, “application of”, and variances thereof, refer to the placement of a tourniquet to an extremity of an individual in order to restrict blood flow through the extremity. The phrase “restrict blood flow”, and variances thereof, means to reduce the flow of blood and other fluids and solids through the blood vessels of the circulatory system of an individual to a point below the normal flow rate for that individual at the attachment point of the tourniquet to an extremity. The terms “individual” and “patient” herein refer to any human individual or other mammal. Herein, “failure” of the safety member refers to the desired breaking apart, separation, tearing, splitting, decomposing, decompressing, creeping or dissolving of the safety member itself, and/or the detachment of the safety member from the first compression section and/or the second compression section. The terms “unused tourniquet” and “new tourniquet” refer to a tourniquet that is ready to be applied to an extremity of a patient. The term “disposable” refers to one time use of the tourniquet.

In one aspect, the present application provides a disposable tourniquet effective to automatically fail at a predetermined time following application of the tourniquet to a patient.

In another aspect, the present application provides a tourniquet comprising a self-releasing means effective to fail at a predetermined time following application of the tourniquet to a patient.

In another aspect, the present application provides a tourniquet comprising a self-releasing means wherein the tourniquet may be configured to trigger a chemical or physical reaction effective to cause the failure of the self-releasing means to detach the tourniquet from a patient.

In another aspect, the present application provides a tourniquet operationally configured to prevent ischemia without any external influence following application of the tourniquet to an extremity of a patient.

### Brief Description of the Tourniquet

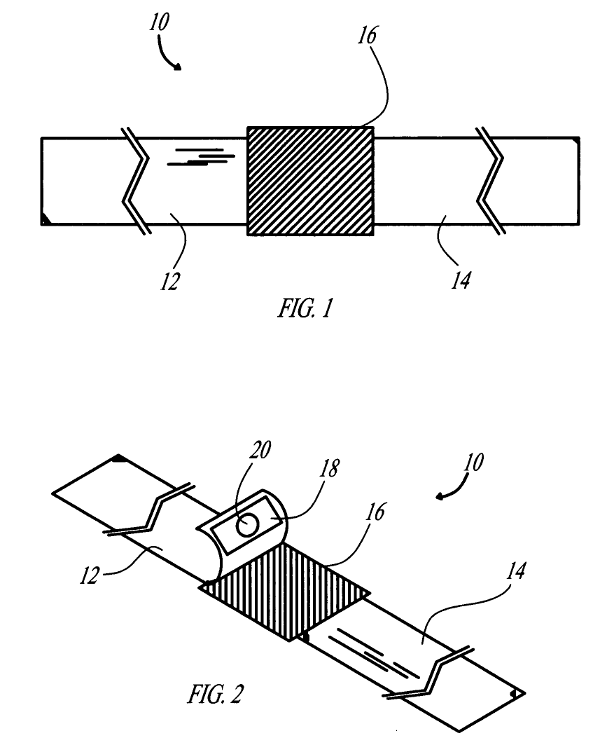

The Figures of the drawings, and particularly FIG. 1, disclose a self-releasing tourniquet 10. The tourniquet 10 suitably comprises a first end and a second end comprised of a compression means and a releasing means positioned therebetween. The compression means suitably comprises first and second compression sections 12 and 14, while the releasing means suitably comprises a safety member 16 configured to fail at a predetermined time during operation of the tourniquet 10. In one embodiment, the safety member 16 may be configured to fail without any external influence. In another embodiment, the tourniquet 10 may include a chemical means for causing the failure of the safety member 16 during operation.

Suitably, the safety member 16 interconnects the first and second compression sections 12 and 14 so that the first and second compression sections 12 and 14 are attached at opposing ends of the safety member 16. As shown in FIG. 2, the tourniquet 10 may further include an adhesion means (hereafter “adhesion member 18”) located on the first compression section 12, and an activation means (hereafter “activator 20”) located near, on, or otherwise surrounded by the adhesion member 18. As discussed in detail below, the activator 20 is suitably effective to trigger or otherwise cause the failure of the safety member 16 during operation of the tourniquet 10.

Accordingly, the tourniquet 10 comprises a length necessary for the tourniquet 10 to be applied to the extremity of an individual. In a simplified embodiment, the tourniquet 10 comprises a length necessary for the tourniquet 10 to be tied around an extremity of an individual. As explained below, the sum of the lengths of the compression sections 12 and 14 is suitably greater than the length of the safety member 16—although not necessarily required. In addition, although the tourniquet 10 is not necessarily limited to a particular configuration, the safety member 16 suitably includes a width and thickness similar to compression sections 12 and 14 thereby forming a substantially uniform and continuous tourniquet 10 of a desired length.

In addition, the compression sections 12 and 14 and safety member 16 have a collective length necessary to self-activate the tourniquet 10—either in a stretched or non-stretched state. Thus, the first compression section 12 and second compression section 14 may comprise elongated members of either substantially equal non-stretched lengths, substantially unequal non-stretched lengths, substantially equal stretched lengths, or substantially unequal stretched lengths effective for the application of the tourniquet 10 to a target extremity.

Not only do the compression sections 12 and 14 comprise a collective length necessary for application of the tourniquet 10 to an extremity, but the compression sections 12 and 14 also comprise a collective length necessary for adequate compression to restrict blood flow through an extremity. Therefore, the compression sections 12 and 14 are suitably constructed from one or more elastic or stretchable materials effective to compress the extremity during operation of the tourniquet 10. Suitably materials include, but are not necessarily limited to elastic rubber and stretchable fabric materials. Suitable stretchable fabric materials include, but are not necessarily limited to, elasticized cloth, synthetic fabrics and the like. Herein, any combination of compression section materials may be used. In one embodiment, each of the compression sections 12 and 14 may be constructed from similar materials. In an alternative embodiment, each of the compression sections 12 and 14 may be constructed from dissimilar materials.

In a particularly advantageous embodiment, the tourniquet 10 comprises a first compression section 12 and second compression section 14 constructed from flat elastic rubber band type material capable of being tied together. Alternatively, the first compression section 12 and the second compression section 14 may be constructed from elastic tubular rubber, i.e., surgical hose. In addition, it is herein contemplated that other non-flat and non-tubular elastic rubber pieces be utilized as compression sections 12 and 14. Suitable elastic rubber band type material can be acquired from the following commercial source: Home Care Delivered, Inc., located in Glen Allen, Va.

Although the tourniquet 10 may be built to scale, a suitable tourniquet 10 intended for humans includes a first compression section 12 and a second compression section 14 constructed from elastic rubber, each section having (1) a length from about 10 cm to about 30 cm (from about 3.94 inches to about 11.81 inches), (2) a width from about 1.5 cm to about 3.0 cm (from about 0.59 inches to about 1.18 inches), and (3) a thickness from about 0.1 mm to about 2.0 mm (from about 0.004 inches to about 0.079 inches). In a particularly advantageous embodiment, the first compression section 12 and second compression section 14 are constructed from flat elastic rubber each section having (1) a length of about 19 cm (about 7.48 inches), (2) a width of about 2.54 cm (of about 1.0 inches), and (3) a thickness of about 1.0 mm (of about 0.04 inches).

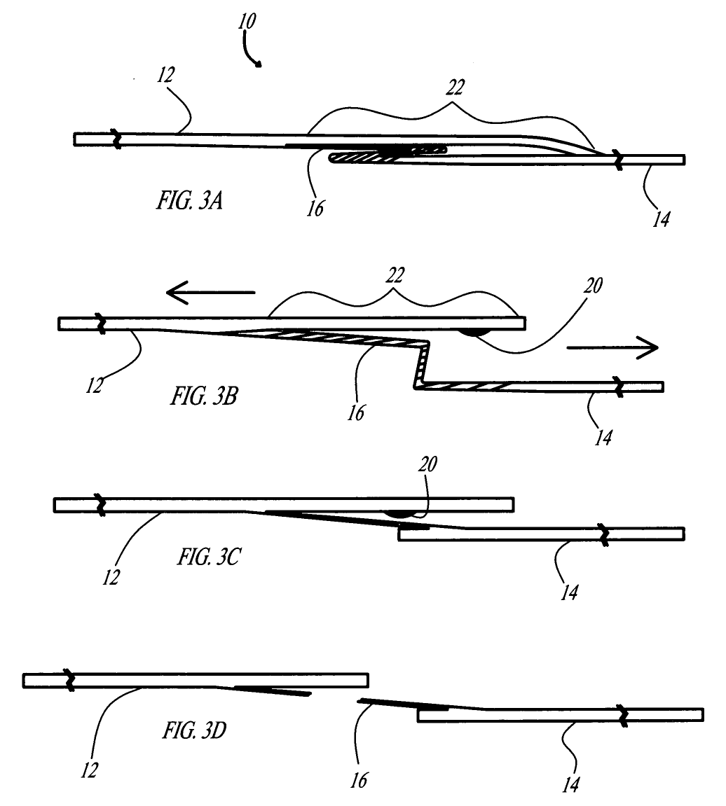

As FIG. 1 illustrates, the safety member 16 is suitably disposed at a non-terminal point of the tourniquet 10 interconnecting the first compression section 12 and the second compression section 14. Suitably, the safety member 16 is constructed from a material effective to securely affix to each of the first compression section 12 and the second compression section 14 during application of the tourniquet 10. In addition, the safety member 16 is suitably constructed from a flexible or semi-flexible material that can be folded or bent—as shown in FIGS. 3A and 3B. As previously stated, the safety member 16 may be constructed from a material having a width and thickness substantially similar to the corresponding compression sections 12 and 14. In the alternative, the safety member 16 may be constructed from a material dissimilar in width and/or thickness from the corresponding compression sections 12 and 14. The safety member 16 may be affixed to the compression sections 12 and 14 via a threaded connection, adhesion, melting, tape, and combinations thereof.

In relation to the above described compression sections 12 and 14, a suitable safety member 16 includes a length from about 2.0 cm to about 5.0 cm (about 0.79 inches to about 1.97 inches). In a particularly advantageous embodiment, a suitable safety member 16 comprises a length of about 3.75 cm (about 1.48 inches). As stated previously, a suitable safety member 16 includes a width substantially similar to the width of the compression sections 14 and 16. Likewise, the safety member 16 may include any thickness in comparison to corresponding compression sections 14 and 16. As stated previously, a suitable safety member 16 comprises a thickness substantially similar to the thickness of the compression sections 12 and 14. As discussed in more detail below, the safety member 16 is suitably constructed from any material effective to fail at a predetermined time following application of the tourniquet 10.

With reference again to FIG. 2, the tourniquet 10 suitably includes at least one activator 20 effective to trigger the failure of the safety member 16 following application of the tourniquet 10. Suitably, the activator 20 is comprised of one or more chemical substances effective to trigger the failure of the safety member 16 upon contact. Thus, it is herein contemplated that contact between the activator 20 and the safety member 16 causes a chemical reaction that leads to the eventual failure of the safety member 16.

In another embodiment, the safety member 16 may be constructed from one or more creep materials effective to fail based on the amount of tension applied to the safety member 16 by each of the compression sections 12 and 14 during application of the tourniquet 10. For example, as the tourniquet 10 is applied to an extremity of an individual, the compression sections 12 and 14 can be stretched in opposing directions placing tension upon the safety member 16 thereby causing creep deformation, i.e., failure of the safety member 16 at a predetermined stress level. Suitable creep materials include, but are not necessarily limited to plastics, rubbers, nylon, rayon, and foam fabric.

In an embodiment incorporating an activator 20, the safety member 16 material(s) to be used depend on the corresponding chemical substances used to form the activator 20, and vice versa. In one exemplary embodiment, wherein the safety member 16 is constructed from a corn starch based material, the corresponding activator 20 may include for example, amylase. In another exemplary embodiment, including a safety member 16 constructed from a protein based material, the corresponding activator 20 may include for example, protienase. In another exemplary embodiment, wherein the safety member 16 is constructed from a cellulose based material such as paper, the corresponding activator 20 may include for example, a water based material, a water based solution, a water based gel, cellulose, and combinations thereof. In another exemplary embodiment, wherein the safety member 16 is constructed from a fibrin based material, the corresponding activator 20 may include for example, fibrinogen, plasminogen, and combinations thereof. In another exemplary embodiment, wherein the safety member 16 is constructed from a keratin based material, the corresponding activator 20 may include for example, an aqueous alcoholic solution including ethanol, propanol, ammonium thioglycate, barium sulfate, calcium thioglycate, ethanolamine thioglycate, mercaptopropionic acid, potassium thioglycate, sodium thioglycate, thioglycerine, thioglycic acid, thioacetic acid, and combinations thereof.

It is herein contemplated that the safety member 16 may be constructed from materials other than those materials described above. Likewise, it is herein contemplated that the activator 20 may comprise chemical substances other than those chemical substances described above. In addition, a preferred activator 20 is comprised of one or more chemical substances not harmful to at least the skin, eyes and mucous membranes of an individual, or in the alternative, not harmful at the concentrations contemplated herein.

The amount of activator 20 effective to cause failure of the safety member 16 may vary depending on the type of safety member 16 and activator 20 used. For example, in an embodiment where the safety member 16 is constructed from corn starch and has the parameters as listed in Table 1 below, an activator 20 comprised of amylase may be present in an amount from about 0.1 ml to about 2.5 ml (from about 0.0034 fluid ounces to about 0.085 fluid ounces):

TABLE 1

| | |
|---|---|
| LENGTH | about 3.75 cm (about 1.48 inches) |
| WIDTH | about 2.54 cm (about 1.00 inches) |
| THICKNESS | about 1.50 mm (about 0.06 inches) |
| MASS | about 2.00 grams (about 0.07 ounces) |

For the purposes of the present application, the adhesion member 18 suitably comprises an attachment or adhesion means sufficient to maintain the adherence of the first compression section 12 to the second compression section 14 prior to application of the tourniquet 10, while also displaying properties necessary for the separation of the first compression section 12 from the second compression section 14 either (1) as the tourniquet 10 is being applied, or (2) following application of the tourniquet 10.

In one exemplary embodiment, the first compression section 12 can be adhered to the second compression section 14 via an adhesion member 18 comprising one or more mechanical means for adhering sections 12 and 14. In one such implementation, the first compression section 12 can be interwoven to the second compression section 14 using one or more threadlike materials operationally configured to join the first compression section 12 to the second compression section 14. Suitable threadlike materials include for example, thread and other thin fibers. In another implementation, the first compression section 12 can be joined to the second compression section 14 using tape. Suitable tape materials include for example, transparent tape, masking tape, duct tape, electrical tape and surgical tape. In still another embodiment, the first compression section 12 can be attached to the second compression section 14 via fabric hook-and-loop fasteners. An exemplary fabric hook-and-loop fastener includes Velcro®. In still another embodiment, the activator 20 may be enclosed within a thin membrane housing, whereby the adhesion member adheres the housing to the second compression section 14, thus tearing open the housing upon application of the tourniquet 10.

In another exemplary embodiment, the first compression section 12 can be adhered to the second compression section 14 via an adhesion member 18 comprising one or more adhesive substances or bonding type substances. As FIG. 3A illustrates, one or more adhesion members 18 may be located on a section of the first compression section 12 that is operationally configured to extend beyond the safety member 16 to contact the second compression section 14—prior to the application of the tourniquet 10. Suitable adhesives include for example, natural adhesives, synthetic adhesives, drying adhesives, thermoplastic adhesives, and reactive adhesives. Natural adhesives may include for example, natural gums and plant resins. Synthetic adhesives may include for example, adhesives based on elastomers, thermoplastic, and thermosetting adhesives. Drying adhesives may include for example, rubber cements and glues such as white glue.

Depending on the particular chemical substance(s) that make up the activator 20, the adhesion member 18 can be configured to encase the corresponding activator 20 (as shown in FIG. 2) or the activator 20 can be set apart from the corresponding adhesion member 18. In the embodiment of FIG. 2, where the activator 20 is encased within the adhesion member 18, the adhesion member 18 suitably forms a perimeter around the activator 20 to seal the activator 20 when the first compression section 12 is adhered to the second compression section 14 prior to application.

To operate effectively, the activator 20 is suitably prevented from contacting the safety member 16 until actual application of the tourniquet 10 to a patient's extremity. With particular reference to FIG. 3A, a first end of the safety member 16 is suitably attached to the first compression section 12 at a point effective to form an overlapping section 22 of the first compression section 12, wherein the overlapping section 22 is operationally configured to extend beyond the safety member 16 a distance effective to contact at least part of the overlapping section 22 to at least part of the second compression section 14. In this position, the activator 20 is set apart from the safety member 16 until application of the tourniquet 10.

Other embodiments of the tourniquet 10 can be incorporated without deviating from the scope of the subject invention. For example, one or more adhesion members 18 and one or more activators 20 can be located on an exterior surface of the second compression section 14, rather than the first compression section 12, wherein the one or more adhesion members 18 are effective to releasably attach the overlapping section 22 to the second compression section 14. In addition, a sleeve may be added to envelope at least part of the tourniquet 10 in order to maintain the activator 20 against the safety member 16 following application.

### Operation of the Tourniquet

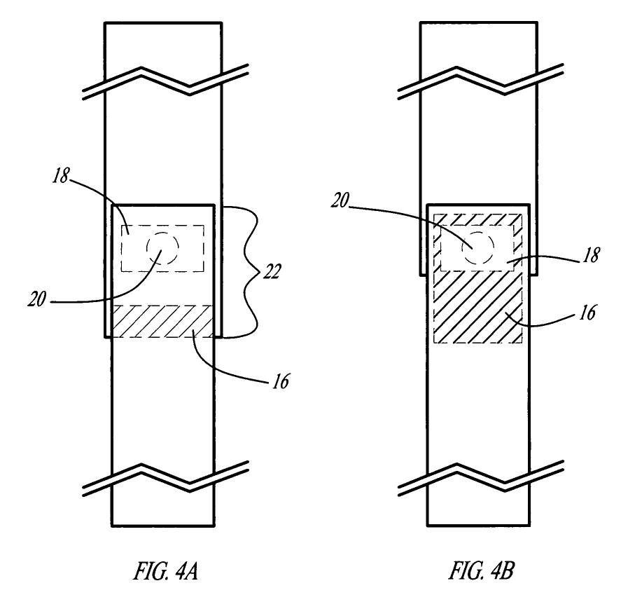

Referring now to FIGS. 3A-3D, there is illustrated, in sequence, a simplified tourniquet 10 prior to application (FIG. 3A), during application (FIGS. 3B and 3C), and following the release of the tourniquet 10 from an extremity of an individual (FIG. 3D). With initial reference to FIG. 3A, a tourniquet 10 is provided including a safety member 16 that is folded upon itself in a manner effective for the overlapping section 22 to extend beyond the safety member 16 to an attachment position with the second compression section 14. With reference to FIGS. 3B and 3C, as the tourniquet 10 is applied to an extremity of an individual the first and second compression sections 12 and 14 can be directed in opposing directions, i.e., pulled or stretched in opposing directions, and around the extremity—as shown by the direction of the arrows in FIG. 3B. As the first and second compression sections 12 and 14 are directed in opposing directions, i.e., as the tourniquet 10 is being stretched and tied around an extremity, the opposing ends of the safety member 16 are also pulled in opposing directions effectively unfolding the safety member 16 to an open position as shown in FIG. 3C. As the safety member 16 opens, the overlapping section 22 is directed toward the safety member 16 wherein the activator 20 located on the interior surface of the overlapping section 22 contacts the safety member 16 to trigger the failure of the safety member 16 and the eventual release of the tourniquet 10. FIGS. 4A and 4B correspond to FIGS. 3A and 3C, respectively.

Although the tourniquet 10 can be configured to fail at any point in time following application of the tourniquet 10, in an attempt to prevent or avoid ischemia or other injury to an individual, a suitable self-releasing or self-decompressing time is up to about ten (10) minutes following application of the tourniquet 10. Thus, any chemical reaction between the activator 20 and the safety member 16 suitably causes the failure of the safety member 16 within ten minutes following contact of the activator 20 with the safety member 16. In addition, the releasing means and activation means can be altered in type and quantity to adjust the time in which the tourniquet decompresses following application of the tourniquet. It is also contemplated herein that during application of the tourniquet 10, the safety member 16 initially assists the compression sections 12 and 14 with the restriction of blood flow until failure of the safety member 16 or shortly before failure of the safety member 16.

The embodiments described above will be better understood with reference to the following non-limiting example, which is illustrative only and not intended to limit the present application to a particular embodiment.

### EXAMPLE 1

In one non-limiting example of the tourniquet 10, where the tourniquet 10 is to be applied to an arm of a patient, the arm having a circumference of about 30.48 cm (about 12 inches), the first compression section 12 and second compression section 14 are comprised of flat elastic rubber bands each having (1) a length of about 19.0 cm (about 7.48 inches), (2) a width of about 2.54 cm (about 1.0 inch), and a thickness of about 1.00 mm (about 0.04 inches). The safety member 16 is a corn starch based material comprising a width and thickness similar to the compression sections 12 and 14, and comprising a length of about 3.81 cm (about 1.5 inches). The activator 20 comprises amylase present in an amount of about 1.5 ml (about 0.05 fluid ounces). The adhesion member 18 is a piece of transparent tape attaching the overlapping section 22 to the second compression section 14.

As will be understood by those of ordinary skill in the art, and others, many modifications may be made without departing from the spirit and scope of the invention. The embodiments described herein are meant to be illustrative only and should not be taken as limiting the invention, which is defined in the following claims.

I claim:

1. A self-releasing tourniquet comprising:
a compression means; and
a releasing means positioned at a non-terminal point along the length of the compression means, thereby dividing the compression means into two parts;
whereby the releasing means is configured to fail at a predetermined time following application of the tourniquet.

2. The tourniquet of claim 1, further comprising an activation means located on the compression means and configured to trigger the failure of the releasing means.

3. The tourniquet of claim 2, further comprising an adhesion means located on the compression means and configured to adhere the two parts of the compression means prior to application of the tourniquet.

4. The tourniquet of claim 2, wherein the activation means is comprised of one or more chemical substances effective to trigger the failure of the releasing means upon contact.

5. The tourniquet of claim 2, wherein the releasing means is constructed from a cellulose based material.

6. The tourniquet of claim 5, wherein the activation means is a water based material.

7. The tourniquet of claim 2, wherein the releasing means is constructed from a corn starch based material.

8. The tourniquet of claim 7, wherein the activation means is amylase.

9. The tourniquet of claim 3, wherein the adhesion means is selected from the group consisting of natural adhesives, synthetic adhesives, drying adhesives, thermoplastic adhesives, and reactive adhesives.

10. The tourniquet of claim 3, wherein the adhesion means is comprised of a tape material.

11. The tourniquet of claim 1 wherein the releasing means is constructed from one or more creep materials.

12. The tourniquet of claim 11, wherein the one or more creep materials are selected from the group consisting of plastics, rubbers, nylon, rayon, and foam fabric.

13. The tourniquet of claim 1, wherein the compression means is comprised of elastic rubber band type material.

14. The tourniquet of claim 1, wherein the tourniquet is disposable.

15. A self-releasing tourniquet comprising:
a first compression section;
a second compression section;
a foldable releasing means attached to the first compression section at a first end and attached to the second compression section at a second end;
an adhesion means for adhering the first compression section to the second compression section prior to application of the tourniquet; and
an activation means located on the first compression section and configured to contact the releasing means upon application of the tourniquet, the activation means being effective to trigger the failure of the releasing means.

16. The tourniquet of claim 15, further comprising a sleeve.

17. A method of disabling a tourniquet during use without any external influence, the method comprising the following steps:
providing a tourniquet comprising a compression means, a releasing means, an adhesion means, and an activation means;
applying the tourniquet to an extremity of a patient thereby contacting the activation means to the releasing means;
forming a reaction between the activation means and the releasing means which results in the failure of the releasing means; and
decompressing the tourniquet from the extremity of the patient.

18. The method of claim 17 wherein the activation means is comprised of one or more chemical substances effective to trigger the failure of the releasing means upon contact.

19. The method of claim 17 wherein the tourniquet decompresses within ten minutes following application of the tourniquet.

20. The method of claim 17 wherein the releasing means and activation means can be altered to adjust the time in which the tourniquet decompresses following application of the tourniquet.
